(19) United States
(12) Patent Application Publication
(10) Pub. No.: US 2007/0224165 A1
(43) Pub. Date: Sep. 27, 2007

(54) Neuroprotective Effects of Gly-Pro-Glu Following Intravenous Infusion

(75) Inventors: Jian Guan, Auckland (NZ); Gregory Brian Thomas, Western Australia (AU); David Charles Batchelor, Auckland (NZ); Peter David Gluckman, Auckland (NZ)

Correspondence Address: BORSON LAW GROUP, PC, 1320 WILLOW PASS ROAD, SUITE 490, CONCORD, CA, 94520-5232, US

(73) Assignee: Neuren Pharmaceuticals Limited, Auckland (NZ)

(21) Appl. No.: 10/574,280

(22) PCT Filed: Oct. 22, 2004
(86) PCT No.: PCT/US04/35165

§ 371 Date: Mar. 9, 2007

Related U.S. Application Data
(60) Provisional application No. 60/513,851, filed on Oct. 23, 2003.
(60) Provisional application No. 60/515,397, filed on Oct. 28, 2003.
(60) Provisional application No. 60/553,688, filed on Mar. 16, 2004.

(51) Int. Cl. A61K 38/06

(52) U.S. Cl. 424/85.2; 424/130.1; 424/85.4; 514/12; 514/18; 514/312; 530/331

(57) ABSTRACT

Gly-Pro-Glu (GPE) is rapidly metabolized in vivo. We found that GPE infusion elicits potent and consistent neuroprotection in all brain regions examined, and in certain embodiments, the effects were greater than those of a bolus injection followed by infusion (“loading dose/infusion”). GPE reduced apoptosis in the hippocampus and inhibited microglial proliferation and prevented the injury-induced loss of astrocytes and improved long-term somatofunction. GPE after infusion showed a broad effective dose range (0.3-30 mg/kg/h) and had a surprisingly extended window of treatment efficacy, permitting its use from 1 to at least as late as 24 h after neural injury. We also found that neuroprotective effects of acute GPE administration were prolonged and therefore capable of being used effectively to treat a variety of neurodegenerative conditions, even when administered after a neural injury. Thus, GPE can be an effective neuroprotective agent used either alone or co-administered along with other neuroprotective agents, antiinflammatory agents or peptidase or protease inhibitors. Compositions of GPE and protease and/or peptidase inhibitors are provided.

### PRIORITY CLAIM

This application claims priority to U.S. Provisional Patent Application Ser. No. 60/513,851 titled “Pharmacokinetics of GPE and Methods of Administration,” David Charles Batchelor, Gregory Brian Thomas, Peter D. Gluckman and Bernhard Hermann Heinrich Breier, Inventors, filed Oct. 23, 2003 (Attorney Docket No: NRNZ 1052 US0 DBB), to U.S. Provisional Patent Application Ser. No. 60/515,397, titled “Pharmacokinetics of GPE and Methods of Administration,” David Charles Batchelor, Gregory Brian Thomas, Peter D. Gluckman and Bernhard Hermann Heinrich Breier, Inventors, filed Oct. 28, 2003 (Attorney Docket No: NRNZ 1052 US1 DBB), and to U.S. Provisional Patent Application Ser. No. 60/553,688, titled “Neuroprotective Effects of Gly-Pro-Glu Following Intravenous Infusion,” Jian Guan, Gregory Brian Thomas, David Charles Batchelor, Bernhard Hermann Heinrich Breier, and Peter D. Gluckman Inventors, filed Mar. 16, 2004 (Attorney Docket No: NRNZ 1052 US2 DBB). Each of the above applications is incorporated herein fully by reference.

### BACKGROUND

Acute ischemic brain injury is one of the major causes of death and long-term disability in adult life. Currently it can be treated by thrombus to enhance brain perfusion if patient can be registered to the clinic within 3 h of the onset of stroke. Neuroprotection has been considered to be another mechanism for treating acute ischemic brain injuries (Lutsep and Clark, 1999). It has been well documented that the majority of neurons die several hours, even days following ischemic injuries, such as stroke or neurological complications associated with open heart surgery (Coimbra et al. 1996; Beilharz et al. 1995; Gallyas et al. 1992; Hsu et al. 1994; Jeon et al. 1995). This evolution of cell loss is progressive due to the initiation of the programmed cell death pathways, which offers a window of opportunity for treatment intervention.

Insulin-like growth factor-1 (IGF-1) occurs naturally in the central nervous system (CNS) (Baskin et al. 1988), plays an important role in CNS development and acts as a survival and differentiation factor for both neuronal and glial cells (Aberg et al. 2000; O'Donnell et al. 2002). It has been well documented that IGF-1 can prevent neuronal death from several forms of ischemic injury in both the mature (Gluckman et al. 1992; Guan et al. 1996; Guan et al. 1993) and developing brain (Johnston et al. 1996; Galli et al. 1995). An anti-apoptotic role for IGF-1 has also been demonstrated in vitro (Yin et al. 1994). However, clinical application of IGF-1 has been problematic due to IGF-1's limited capability to cross the blood-brain barrier (BBB) and its potential for mitogenic and metabolic effects.

Sara et al. (1989) first suggested that IGF-1 can be naturally cleaved into des-N (1-3)-IGF-1 (des-IGF-1) and the N-terminal tripeptide, glycine-proline-glutamate (“Gly-Pro-Glu;” also referred to as “GPE”) in a process mediated by an acid protease (Yamamoto and Murphy, 1994; Sara et al. 1989; Yamamoto and Murphy, 1995). Without interacting with IGF-1 receptor, GPE has been demonstrated to be able to stimulate dopamine and acetylcholine release in vitro (Nilsson-Håkansson et al. 1993). We have previously demonstrated that intracerebroventricular administration of GPE can protect neurons after hypoxic-ischemic (HI) brain injury, particularly in the cerebral cortex and the CA1-2 sub-regions of the hippocampus (Guan et al. 1999). Because it would be much easier to treat patients using intravenously (i.v.) administered agents compared to administration into the cerebral ventricles, development of effective i.v. treatments are highly desirable. Because of its small molecular size, GPE may cross the compromised blood-brain barrier (BBB) after ischemic injury. Thus, one objective of the present invention is to develop effective i.v. therapy to mitigate degeneration or death of neurons.

### SUMMARY

With the recent development of an immunological assay for GPE, we have discovered that GPE is rapidly degraded in the circulation in animals. In spite of its rapid degradation, however, intravenous (i.v.) administration of GPE exhibits neuroprotective effects similar to those observed after direct intraventricular administration. In certain embodiments of this invention, GPE can be injected directly into the circulation of an animal and can decrease or prevent neural cell death. In other embodiments, an i.v. bolus of GPE can be administered without any subsequent infusion. In further embodiments, an i.v. bolus can be followed by a sustained intravenous infusion of GPE. In still other embodiments, a sustained intravenous injection can be used without any prior bolus injection. We have unexpectedly found that sustained i.v. administration of GPE can have more pronounced neuroprotective effects when administered without a preceding bolus injection.

We have also unexpectedly found that GPE can protect neurons from death or degeneration even if administered after the insult that results in the neuronal death or degeneration. In some embodiments, GPE can be administered up to 24 hours after the insult.

We have also unexpectedly found that administration of GPE over a short time period can have prolonged therapeutic benefits, and can result in at least partially reversing the progressive neurodegeneration that typically occurs after a neuronal insult. In some embodiments, a short-term administration of GPE can have neuroprotective effects that persist for 30 days after the insult and after treatment. These findings are completely unexpected based on the short half-life of GPE or any prior art.

In additional aspects, one can use peptidase and/or protease inhibitors to decrease degradation of GPE in the plasma and therefore increase or prolong the effects of GPE.

In additional aspects, compositions are provided comprising GPE and a protease inhibitor.

### BRIEF DESCRIPTION OF THE FIGURES

This invention is described with reference to particular embodiments thereof. Other aspects and features of the invention can be appreciated from reviewing the Figures, in which:

FIG. 1A depicts a graph of plasma concentration of GPE following a single i.v. bolus injection of 3 mg/kg GPE given 2 h after HI injury in adult rats; n=10 animals.

FIG. 1B depicts levels of either vehicle or GPE in the CSF after a 4 h i.v. infusion of 3 mg/kg/h GPE in normal and HI injured rats. HI injured rats were treated 1-5 h after injury. Data are presented as mean±SEM. n=6-9 animals per group. *P<0.05 compared with the HI injured vehicle control group.

FIG. 2A depicts a graph of effects of vehicle or GPE on long-term neuronal survival in different regions of the brain of rats. Animals were infused with 3 mg/kg/h GPE (n=6) or vehicle (n=6) 1-5 h after HI brain injury. The long-term histological and behavioral outcomes were examined 21 days after HI injury. Data are presented as mean±SEM; ***p<0.001 compared to the vehicle control group.

FIG. 2B depicts a graph of somatofunctional recovery in the animals also shown in FIG. 1A.

FIG. 3A depicts a graph of effects of vehicle or GPE on apoptotic cells in the injured right hippocampus detected with an antibody to caspase-3. Data are presented as mean±SEM. n=11-16 animals per group. Animals were infused with 3 mg/kg/h GPE or vehicle 1-5 h after HI brain injury and killed 4 days later. **P<0.01, ***P<0.001 compared with the vehicle control group.

FIG. 3B depicts a graph of effects of vehicle or GPE on apoptotic cells in the injured right hippocampus detected with TUNEL staining in the same animals as shown in FIG. 3A.

FIG. 4 depicts graphs of numbers of cells in the hippocampus of rats treated with vehicle or GPE. Data are presented as mean±SEM. n=13-14 animals per group. Animals were infused with 3 mg/kg/h GPE or vehicle 1-5 h after HI brain injury and killed 4 days later. *P<0.05, ***P<0.001 compared with vehicle control group. # P<0.05 compared with the uninjured left side.

FIG. 4A depicts a graph of effects of vehicle or GPE on isolectin-B4 positive microglia.

FIG. 4B depicts a graph of effects of vehicle or GPE on PCNA positive cells.

FIGS. 4C and 4D depict graphs of effects of vehicle or GPE on GFAP positive astrocytes in the hippocampus.

FIG. 5A depicts a graph of the neuroprotective effect of GPE given either without a prior bolus injection (left column) or after a bolus injection of 3 mg/kg.

FIG. 5B depicts a graph of the effects of bolus injection (left pair of columns) or bolus plus infusion (right pair of columns) of either vehicle (left column of each pair) or GPE (right column of each pair).

FIG. 6 shows pharmacokinetics in plasma in vivo of GPE (30 and 100 mg/kg i.v. bolus). A sharp increase of GPE levels in the plasma was seen immediately (1 min) after either 30 (40±10.8 μg/ml) (FIG. 6A) and 100 (689±125 μg/ml) (FIG. 6B) bolus i.v. injection compared to a baseline of 0.01±0.002 μg/ml. The levels rapidly reduced to baseline with a half-life of 4.95±0.43 min. (Data in parenthesis is mean±SE of 6 repeats).

FIG. 7 shows HPLC analysis of the levels of the GPE, Glu, Gly, Pro and Gly-Pro in plasma at 1, 2 and 8 min following i.v administration of 30 mg/kg GPE. GPE had a retention time of 72 min while the di-peptide Gly-Pro was detected as a broad peak with a retention time of approximately 88 min. Glutamate (Glu) Glycine (Gly), Proline (Pro) eluted at 17.7, 37.5 and 75.2 min respectively.

FIG. 8 depicts a graph showing neuroprotective effects in animals exposed to GPE 24 hours after mild HI injury.

FIG. 9 depicts a graph showing neuroprotective effects in another group of animals exposed to GPE 24 hours after serious HI injury.

### DETAILED DESCRIPTION

In certain aspects, this invention includes methods for determining the amount of GPE in the circulation of an animal. A radioimmunoassay procedure for measuring GPE has been described in PCT International Application Serial No: PCT/US02/08195, in U.S. patent application Ser. No. 10/100,515, filed Mar. 14, 2002, titled “Anti-GPE Antibodies, Their Uses and Analytical Methods for GPE,” Gregory Brian Thomas, Bernhard Hermann Heinrich Breier and David Charles Batchelor, inventors, (Attorney Docket No: NRNZ 1016 US1 DBB) and in United States Utility Patent Application titled “Anti-GPE Antibodies, Their Uses, and Assays for Weakly Immunogenic Molecules,” Inventors: Gregory Brian Thomas, Bernhard Hermann Heinrich Breier and David Charles Batchelor, filed concurrently (Attorney Docket No: NRNZ 1016 US2 DBB). Each of the above applications is herein incorporated fully by reference.

GPE has a Short Half-Life in Plasma In Vivo

Using such an assay, we have found that GPE is removed from the circulation with a half-life of between about 1 to 2 minutes. Although the mechanism for this removal is not certain, proteases and peptidases known to be present in the circulation may be responsible for degrading the GPE. Regardless of the mechanism for its removal, implications for intravenous therapy are significant.

First, with rapid removal from the circulation, neuroprotective amounts of GPE can be maintained by infusion of the agent into the circulation. In other embodiments, a relatively small bolus of GPE can be followed by a sustained infusion to produce even greater neuroprotective effects than those produced by bolus alone. In still other embodiments, a sustained infusion of GPE without an initial bolus can result, surprisingly, in even larger neuroprotective effects than those produced by bolus followed by sustained infusion.

Our data demonstrated central penetration of GPE because a significant elevation of GPE in the CSF following 4 h i.v. infusion was only found in HI injured rats. Ischemic injury-induced disruption of the blood brain barrier can be as early as 2-4 h after the injury due to the loss of tight junctions of the endothelium. Mechanisms by which GPE can enter the CSF are not known with certainty. However, according to one hypothesis, activation of matrix metalloproteinases can also disrupt the endothelial basal lamina (Fujimura et al. 1999; Planas et al. 2001). The small molecular size and hydrophilic nature of GPE may make access to the brain problematic. Therefore disruption of basal lamina after ischemic injury (Fujimura et al. 1999) may be associated with uptake of GPE into the brain.

MK-801, a non-competitive NMDA receptor antagonist is a small molecule with well-documented neuroprotective effects in animals. However, ischemic brain injury reduces MK-801 binding to tissues after peripheral administration (Wallace et al. 1992), unlike the injury-associated CNS uptake of GPE. Compared to substances having non-specific access to the CNS, injury mediated central penetration of GPE can provide more specific targeting of the agent to injured regions of the brain and can minimize unwanted interactions with uninjured regions. However, other hypotheses may account for the observations, and we do not intend this application to be limited to any particular mechanism of action.

The degree of neuroprotection of GPE following a single bolus injection was significant, but somewhat variable. This variability may be due to the short half-life of GPE in plasma, which was estimated to be less than 2 min after a single bolus administration in HI injured rats. A rapid breakdown into its major metabolites, glycine, glutamate and proline after a bolus intravenous injection of GPE has also recently reported in the normal rats (Batchelor et al. 2003). GPE is cleaved from IGF-1 by an endogenous protease enzyme (Yamamoto and Murphy 1994), and the short half-life of GPE may be related its susceptibility to rapid proteolysis. Given the need to maintain efficacious blood levels, in turn to sustain a stable central uptake of GPE, continuous infusion appears to be an effective route of administration for GPE treatment.

Intravenous Administration of GPE is Effective

Intravenous infusion of GPE achieved consistently robust neuroprotection in all the brain regions examined, with a broad effective dose range. Tissue damage in the dentate gyrus and the cerebral cortex was completely prevented following the treatment of the most effective dose of GPE (3 mg/kg/h for 4 h).

We surprisingly found that in some cases, sustained infusion resulted in greater neuroprotection than did the same amount of drug infused after a bolus injection. Thus, in these cases, a greater neuroprotective effect was observed after a lower dose of GPE. This result was completely unexpected based on the prior art.

Another practical problem for drug discovery and development in response to acute injury or disease is the recruitment of patients in a timely manner and providing rapid access to therapies. For example, despite the proven efficacy of anticoagulants after acute ischemic stroke, the great majority of patients are not enrolled within three hours (Famularo et al 1998; Fisher and Schaebitz 2000). The majority of compounds demonstrated to be neuroprotective in experimental models have rather short window of opportunity (Fisher and Schaebitz 2000). By “window of opportunity” we mean the period of time after an acute event during which effective therapy can be initiated. A few compounds can be administered later than 6 h after injury, but even those have a reduced efficacy compared to the better effects of early treatment (Mary et al. 2001; Williams et al. 2003). However, we unexpectedly found that GPE treatment can be effective if initiated either 3-7 h, 7-11 h. In fact, administration of GPE during these times showed a similar degree of neuroprotection compared to earlier administration. Further, administration of GPE 24 h after HI injury also had notable neuroprotective effects. Based on these findings, we believe that beneficial effects of GPE may also be obtained even if administration is delayed beyond 24 hours. With a broad effective dose range and an extended window of therapeutic opportunity of GPE, animals subjected to neural injuries can be effectively treated with GPE to diminish the magnitude of neuronal death or degeneration, and can diminish loss of function typically associated with chronic neuronal injury. Even a partial neuroprotective effect may be sufficient to decrease adverse effects of stroke, coronary artery bypass surgery, traumatic neural injury or other neural insults. Thus, even if the most efficacious timing and dosing is not achieved in a particular situation, even a modest degree of neuroprotection can be very beneficial to a patient suffering from neural damage.

Even though the underlying mechanisms involved in this extended window of opportunity for treatment are not known with certainty, one hypothesis is that an initial insult can result in delayed neuronal death. In addition to the well-understood secondary neuronal loss during the first week after injury, the delayed cell death can continue in a progressive, “tertiary” phase over many months (Coimbra et al. 1996; Colbourne et al. 1999; Gallyas et al. 1992; Hsu et al. 1994; Jeon et al. 1995). This time-course of delayed cell death was been previously demonstrated in the current experimental rat system (Guan et al. 2001b). Most experimental studies reported histological endpoints exclusively between 24 hours and a week after initial injury. However, early protection observed with some compounds may not be maintained in the long-term (Fisher and Schaebitz 2000; Gladsrone et al. 2002). Therefore, short-term neuronal outcome does not necessarily reflect overall efficacy of the treatment, and reliance upon only acute studies can be misleading in clinical development by underestimating long-term therapeutic benefit. Current experiments showed i.v. infusion of GPE initiated 1-5 h after the injury improved long-term neuronal outcome examined 21 days following HI injury. The progressive neuronal damage examined 21 days after the injury has been previously reported in this particularly animal model, possibly due to a biphasic effect of endogenous products released during brain injury and recovery (Guan et al. 2001; Sharp et al. 2000).

One of well the recognized pitfalls for the failure in the transformation of results from animal models to humans is that the pre-clinical development is typically evaluated based on histological outcome, whereas clinical trails are evaluated by long-term behavioral or clinical outcomes (Fisher and Schaebitz 2000; Gladsrone et al. 2002).

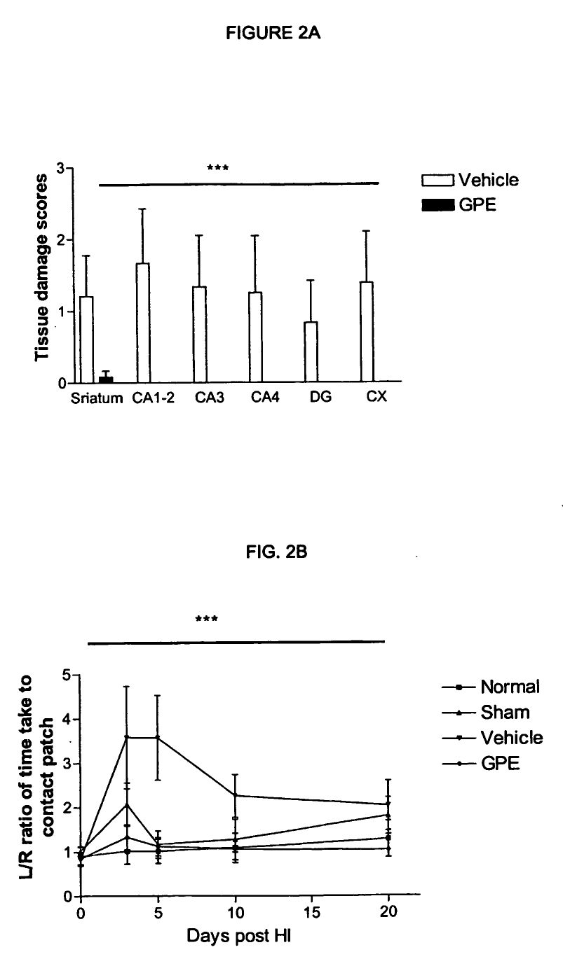

In a previous report, HI injury resulted in unilateral damage within the territory of the middle cerebral artery (Ginsberg and Busto 1989), whose zone of perfusion which is largely associated with somatosensory function (Guan et al. 2001). Neuronal damage in this particular distribution of the cerebral cortex has resulted in significant loss of somatosensory function on the contralateral side to the damaged hemisphere and was most pronounced at the early time points (days 3 and 5). A spontaneous functional recovery was found 10 days after HI injury, probably associated with endogenous production of various growth factors (Yamaguchi et al. 1991; Gasser et al. 1986; Gomez et al. 1992; Klempt et al. 1992). There was no significant recovery in somatosensory function over the period examined in the vehicle treated group. On the other hand, rats showing delayed times to make contact to a patch were also often observed missing the patch during the trial, which may suggest a deficiency of motor co-ordination as the loss of multiple phenotypic neurons in the striatum has been reported after HI injury (Guan et al. 1999). Like treatment with IGF-1 (Guan et al. 2001), treatment with GPE also improved the recovery in somatofunction, with a comparable long-term neuronal outcome (FIGS. 2A & B). We previously reported that the function recovery of IGF-1 is more associated with prevention of progressive neuronal loss rather than the reduction of infarction size (Guan et al. 2001). The current studies showed that tissue cavitation and atrophy was only found in the minority of rats ( 2/6 vehicle treated rats), probably due to a smaller degree of damage in the current experiments. However, the GPE treated group had minimal histological damage and GPE completely prevented the behavioral deficit typically associated with HI.

An acute hypoxic ischemic insult to the brain results in neuronal loss with a mixed pathogenesis. Some cells exhibit necrosis, a morphology recognized for a more rapid evolution of neuronal death initiated by a rupture of cell membranes, whilst other cells are committed to die via a more progressive process initiated by nuclear condensation (e.g. apoptosis). Both forms of neuronal death do not occur immediately following the injury, which provides a window of opportunity for treatment. TUNEL and caspase-3 positive immunostaining have been broadly used as markers for the cells that undergo apoptosis (Snider et al. 1999; Velier et al. 1999). Given that the tissue damage scores currently used assessed a mixture of neuronal necrosis and apoptosis, a similar degree of neuronal damage was found cross the CA1-2, CA3 and CA4 sub-regions of the hippocampus in the vehicle treated group. Interestingly, while an increased TUNEL positive cells were seen mainly in the CA3 sub-region of the hippocampus, the majority of caspase-3 positive cells were located differently in the CA4 sub-regions in the vehicle treated group. Both TUNEL and caspase-3 positive cells were relatively low in the CA1-2 sub-regions. It is thought that, as an execution phase protease, caspase-3 activation (Yakovlev and Faden 2001) leads to the fragmentation of DNA (Springer et al. 2001), where TUNEL can then be positively labeled.

HI injury resulted spatial differences between caspase-3 activation and TUNEL labeling, indicating that a caspase-3 pathway may not necessarily lead to positive TUNEL labeling. This disassociation between the TUNEL and caspase-3 immunoreactivity has also been observed outside of the CNS (Donoghue et al. 1999). Therefore these spatial differences indicated that both caspase-3-dependent and caspase-3-independent pathways were involved in neuronal injury in the hippocampus following HI injury. Our data clearly show that GPE treatment 1-5 h after HI injury significantly reduced the tissue damage, as well as TUNEL and caspase-3 positive cells, suggesting that GPE administration was associated with inhibition of both neuronal necrosis and apoptosis.

A role for glial cells in neuronal damage and recovery has been controversially documented (Kraig, et al. 1995). Our data show that GPE strongly suppressed microglial proliferation, and completely prevented the HI-induced loss of astrocytes. A physiological role for reactive astrocytes has been suggested to be involved in blood brain barrier (BBB) integrity, cell-to-cell communication, intracellular iron-homeostasis, plasticity of neurons, and neurotrophic actions by regulating growth factor metabolism (Kraig et al. 1995). Under physiological conditions, excitatory amino acid release from astrocytes is receptor mediated, whereas injury-induced excitatory amino acid leakage from astrocytes is due to astrocyte swelling (Kraig et al. 1995), which can lead to a damaged homeostasis and can contribute to further neuronal injury. The loss of astrocytes following ischemic injury has also been suggested to be an important part of evolution of tissue infarction (Matsui et al. 2002; Tateishi et al. 2002). Therefore, maintaining astrocyte integrity may be part of the neuroprotective effects of GPE.

Microglial cells are generally believed to have a role in brain inflammation, autoimmune responses and neuronal degeneration (Kraig et al. 1995). Unlike treatment with IGF-1 (Cao et al. 2003), treatment with GPE reduced HI injury-induced isolectin B4-positive microglial cells, probably through inhibiting cell proliferation, because the numbers of PCNA positive cells, a marker of cell proliferation, was also reduced by GPE treatment. Several neuroprotective agents have been identified that have anti-inflammatory properties, such as TGFβ-1 (McNeill et al. 1994), which could be involved in neuroprotection of GPE after HI injury. In contrast to GPE, IGF-1 promotes the proliferation of both astrocytes and microglia after ischemic brain injury (Cao et al. 2003; O'Donnell et al. 2002). This may suggest a different mode of action between GPE and IGF-1 in glial/neuronal interaction.

Administration of GPE by i.v. bolus, i.v. infusion or both i.v. bolus and i.v. infusion can be particularly suitable for treating patients having a neurodegenerative condition. Such conditions include, by way of example only, Huntington's disease, Alzheimer's disease, Parkinson's disease, multiple sclerosis, amyotrophic lateral sclerosis, peripheral neuropathy, spinal muscular atrophy, Creutzfeldt-Jakob disease, AIDS dementia, progressive supranuclear palsy, myelinopathia centralis diffusa (vanishing white matter disease), chronic neurodegenerative disease, Down's syndrome, leukoencephalopathy, hypoxia, ischemia, coronary artery bypass graft (CABG) surgery and Schilder's disease, neuroblastoma, head injury, traumatic brain injury, stroke, reperfusion injury, epilepsy, toxin damage, radiation damage, asphyxia, an inflammatory condition, chronic or acute encephalomyelitis, encephalitis, optic neuritis, transverse myelitis, meningitis, panencephalitis, Devic's disease, progressive multifocal leukoencephalopathy, central pontine myelinolysis and neuromyelitis optica.

The routes of administration described herein are also suitable for administration of other neuroprotective agents. Such agents include, by way of example only, an anti-apoptotic or neuroprotective agent selected from the group consisting of growth factors and associated derivatives (insulin-like growth factor-I [IGF-I], insulin-like growth factor-II [IGF-II], transforming growth factor-β1, activin, growth hormone, nerve growth factor, growth hormone binding protein, IGF-binding proteins [especially IGFBP-3], basic fibroblast growth factor, acidic fibroblast growth factor, the hst/Kfgk gene product, FGF-3, FGF4, FGF-6, keratinocyte growth factor, androgen-induced growth factor, int-2, fibroblast growth factor homologous factor-1 (FHF-1), FHF-2, FHF-3 and FHF-4, keratinocyte growth factor 2, glial-activating factor, FGF-10 and FGF-16, ciliary neurotrophic factor, brain derived growth factor, neurotrophin 3, neurotrophin 4, bone morphogenetic protein 2 [BMP-2], glial-cell line derived neurotrophic factor, activity-dependant neurotrophic factor, cytokine leukaemia inhibiting factor, oncostatin M, an interleukin, α-interferon, β-interferon, γ-interferon, consensus interferon, TNF-α, clomethiazole; kynurenic acid, Semax, tacrolimus, L-threo-1-phenyl-2-decanoylamino-3-morpholino-1-propanol, adrenocorticotropin-(4-9) analogue [ORG 2766], dizolcipine [MK-801], selegiline, a glutamate antagonist, an AMPA antagonist and an anti-inflammatory agent.

Additional neuroprotective agents include glutamate antagonists including NPS1506, GV1505260, MK-801 and GV150526, AMPA antagonist is selected from the group consisting of 2,3-dihydroxy-6-nitro-7-sulfamoylbenzo(f)quinoxaline (NBQX), LY303070 and LY300164 and anti-inflammatory agent selected from the group consisting of an anti-MAdCAM-1 antibody and an antibody against an integrin α4β1 receptor and an integrin α4β7 receptor.

Because GPE is so rapidly degraded in the plasma, the use of peptidase or protease inhibitors can potentiate the effects of and prolong the plasma half-life of GPE. Thus, in additional embodiments, GPE can be administered along with one or more peptidase or protease inhibitors. In some embodiments of this invention, inhibitors of carboxypeptidases, aminopeptidases, peptidyldipeptidases and/or dipeptidases or metalloproteinases can be used. In certain embodiments, one or more inhibitors selected from the group consisting of pepstatin A, leupeptin, bestatin, aprotinin, AEBSF, metalloproteinase inhibitor and E-64 can be co-administered along with GPE to provide heightened and/or prolonged effects.

Additionally, this invention provides new compositions comprising GPE and one or more peptidase inhibitors. Such inhibitors include those listed above as well as others known in the art. Additionally, excipients can be included in a composition comprising GPE and one or more peptidease or protease inhibitors to provide a therapeutic composition suitable for administration to a subject in need thereof.

We have found that GPE exerts robust and potent effects in preventing neuronal injury after HI brain injury. In additional to its injury dependent central penetration and rapid plasma clearance, a broad effective dose range, extended treatment window and long-term functional recovery make GPE a potential candidate to be developed for treating acute ischemic brain injury. Promoting astrocyte survival and inhibiting microglia proliferation may be important for GPE in preventing both neuronal apoptosis and necrosis.

### EXAMPLES

### Example 1

### Animals and Surgery

These studies were approved by the Animal Ethics Committee of the University of Auckland. Every effort was made to minimize animal suffering and to reduce the number of animals used.

Adult male Wistar rats (280-310 g) were obtained from the Animal Resources Unit colony, University of Auckland. Acute brain injury was induced using the modified Levine preparation and has been described previously (Guan et al. 1993). Briefly, the unilateral brain injury was induced by right carotid artery ligation followed by inhalation hypoxia. The right carotid artery was double ligated under general aneathesia (3% halothane/oxygen). After 1 h recovery from the anaesthesia the rats were placed in an incubator where the humidity (90±5%) and temperature (31±0.5° C.) were controlled for a further 1 h. The rats were then exposed to 15 min hypoxia (6±0.2% oxygen). The animals were maintained in the incubator for a further 30 min after the hypoxia before being removed to a holding room.

To permit continuous i.v. infusion, rats in some protocols were chronically catheterized 3 d prior to the experiment as described previously (Thomas et al. 1997). Rats were surgically fitted with an in-dwelling jugular venous catheter and housed individually in metabolic cages. The surgery was conducted under general anaesthesia with 3% halothane/oxygen, where the right jugular vein was exposed and a polyethylene catheter inserted. The catheter were exteriorized and passed out of the cage via a protective stainless steel spring and connected with a fluid-tight swivel joint. This was to allow the animal free movement within the cage. After a 3 d post-surgery recovery period, the catheter was connected to a peristaltic infusion pump to facilitate the infusion of GPE.

### EXPERIMENTAL GROUPS

Experimental groups are shown in Table 1 below.

TABLE 1
Experimental groups

| | Treatment groups | | | | |
|---|---|---|---|---|---|
| | | | HI injured | | |
| Experiments | Non-HI inured | | Vehicle | GPE and doses | |
| Half life in plasma | | | | n = 10 | 15 mg/kg 2 h post HI |
| Central penetration | Vehicle | GPE | n = 9 | n = 9 | 3 mg/kg/h ×4 1-5 h post HI |
| | n = 6 | n = 6 | | | |
| Single bolus | | | n = 14 | n = 14 | 15 mg/kg 2 h post HI |
| Single bolus + iv | | | n = 16 | n = 16 | 3 mg/kg + 3 mg/kg/h ×4 1- |
| infusion | | | | | 5 h post HI |
| Dose dependency | | | n = 7 | n = 10 | 0.03 mg/kg/h 1-5 h post HI |
| | | | n = 11 | n = 9 | 0.3 mg/kg/h 1-5 h post HI |
| | | | n = 16 | n = 16 | 3 mg/kg/h 1-5 h post HI |
| | | | n = 10 | n = 12 | 30 mg/kg/h 1-5 h post HI |
| Window of | | | n = 13 | n = 12 | 3 mg/kg/h 3-7 h post HI |
| treatment | | | n = 16 | n = 16 | 3 mg/kg/h 7-11 h post HI |
| Long-term effects | Normal | Sham | n = 6 | n = 6 | 3 mg/kg/h 1-5 h post HI |
| | n = 6 | n = 6 | | | |

### Pharmacokinetic Studies

Ten HI injured rats were used to determine the half-life of GPE after a single bolus i.v. injection given 2 h after HI injury. Blood samples were collected into heparinised tubes on ice containing protease inhibitor cocktail (Sigma-Aldrich, Sydney, Australia) at 10, and 0 min before, and 1, 2, 4, 8, 16 and 32 min after the i.v. injection of 3 mg/kg GPE (Bachem AG, Basal, Switzerland). The plasma was stored at −80° C. for GPE radioimmunoassay.

In two additional experiments the central penetration of GPE was determined in both normal (n=15) and HI injured rats (n=15). Nine rats from each group of animals were given a continuous 4 h infusion of 3 mg/kg/h GPE. The remaining six rats from each group received a control infusion of 10 mM succinate buffer, pH 6.0 (GPE-vehicle). In HI injured rats GPE treatment was administered 1-5 h after injury. At the end of the 4 h infusion period the animals were anaesthetized and cerebral spinal fluid (CSF) was collected into the tubes containing protease inhibitors from the cisterna magna using a 29-G ultra-fine needle and syringe. The animals were then killed using an overdose of pentobarbital. The CSF samples were stored at −80° C. for GPE assay.

### Example 2

### Treatment Studies

In experiment 1, two groups of 14 HI injured rats received either a single i.v. injection of 15 mg/kg GPE administered 2 h after HI injury or an injection of GPE-vehicle. After 4 d the rats were killed and the brains collected for histological analysis.

In experiment 2, 16 rats were given a single 3 mg/kg i.v. bolus injection of GPE 1 h after HI injury and immediately followed by a continuous 4 h i.v. infusion (3 mg/kg/h) of GPE. The control HI group (n=16) received GPE-vehicle alone. After 4 d the rats were killed and the brains collected for histological and immunohistochemical analysis.

In experiment 3, groups of HI injured rats (n=7-16) received a continuous 4 h i.v. infusion of either 0.03, 0.3, 3 or 30 mg/kg/h GPE beginning 1 h after HI injury. Control HI injured rats received a 4 h infusion of GPE-vehicle alone. The rats were killed and the brains collected for histological analysis 4 days after HI injury.

In experiment 4, groups of HI injured rats (n=12-16) received a continuous 4 h i.v. infusion of 3 mg/kg/h GPE initiated at either 3 or 7 h after HI injury. Control HI injured rats received a 4 h infusion of GPE-vehicle alone corresponding to the relevant treatment window of the experiment (e.g. 3-7 h or 7-11 h after HI injury). The rats were killed and the brains collected for histological analysis 4 days after HI injury.

In experiment 5, 24 rats were used for examining the long-term effects of GPE on both neuronal outcome and functional recovery after HI injury. Rats were divided randomly into 4 groups of normal controls, sham operated, HI treated with either GPE or its vehicle. All rats were habituated with the bilateral tactile tests for 3 sequential days prior to HI injury. Either GPE (3 mg/kg/h) or the vehicle was infused (i.v.) 1-5 h following HI injury. The procedure of the bilateral tactile tests was described previously (Guan et al. 2001b). IGF-1 and vehicle treated rats were tested at 3, 5, 10, and 20 days post insult and the normal controls and sham operated rats were tested in parallel. Each rat had four trials on each day of testing. In each trial, an adhesive label (11 mm²) was applied on the distal radial portion of the left and right forelimbs. The rat was put into a clear perspex observation chamber (190 mm wide×210 mm high×330 mm long). The time taken for the rat to contact each patch was measured (in minutes). The time between trials for each rat on each day of testing was between 5 and 10 minutes, and the order of patch placement was pseudo-random, with the left and right patch each being placed first on 2 of the 4 trials. The trial was ended at 5 minutes if the rat failed to contact to the patch. The time of contact to the patch was considered an indication of somatosensory-motor function of damaged (right) and control (left) hemispheres in the rats. L/R ratio of time taken to contact to the patch was used to quantify the asymmetry between performance on the contralateral limb (left, with potential deficit) and the ipsilateral limb (right, without potential deficit) to the damaged hemispheres. The mean of the ratio for each rat on each day of testing was calculated. Experimenters were blind from the treatmentgroups. Rats were killed 21 days after the HI injury for histological analysis.

### Example 3

### Histology

Histological procedures have been described previously (Guan et al. 1996; Guan et al. 1993). Briefly, 4 d after HI injury and GPE treatment the rats were perfused transcardially under deep anaesthesia with normal saline followed by 10% formalin. The brains were kept in the same fixative for 2 d before being processed using a paraffin procedure. Three coronal (6 μm) sections were cut from the striatum, cerebral cortex and hippocampus, mounted on glass slides and stained with thionine and acid fuchsin.

Dead neurons were identified as those with acidophilic (red) cytoplasm and contracted nuclei (Auer et al. 1985; Brown and Brierley, 1972). Brain tissues with selective neuronal death, cellular reaction and/or pan-necrosis were considered to be damaged (Guan et al. 2000; Markgraf et al. 1993). In additional to the above described pathology, the tissue damage score also included the tissue atrophy and cavitation in the group used for long-term histological examination 21 days after the HI injury. The severity of brain damage in the lateral cortex was assessed using three levels, the dentate gyrus and the CA1-2, 3 and 4 sub-regions of the hippocampus using two levels, and the striatum using one level as following: 0=no damage; 1=<5% tissue damaged; 2=<50% tissue damaged; 3=>50% tissue damaged and 4=>95% damaged (Guan et al. 2000; Lundgren, Smith, and Siesjö, 1992). The average tissue damage scores in different brain regions were used for data analysis (Guan et al. 2000). Any animals that died before the termination of experiments were rejected from the histological analysis. The histology was analyzed by an individual blind to the treatment groups.

Immunohistochemistry

Primary antibodies against glial fibrillary acidic protein (GFAP), isolectin B4, caspase-3, and proliferating cell nuclear antigen (PCNA) were used to mark reactive glial cells and cells undergoing apoptosis and proliferation, respectively.

Immunohistochemical staining was performed in both control and GPE treated HI rats (experiment 2) on paraffin tissues, along with four normal control rats. Coronal sections (6 μm) containing the level of the hippocampus were cut and mounted on chrome-alum coated slides for staining. The sections were deparaffinized in xylene, dehydrated in a series of ethanol and incubated in 0.1 M phosphate buffered saline (PBS). For antigen unmasking (caspase-3 and PCNA staining), sections were heated in 10 mM sodium citrate buffer (pH 6.0) for 1 min at high power. All sections were pretreated with 1% H₂O₂ in 50% methanol for 30 min to quench the endogenous peroxidase activity. Then either 1.5% normal horse serum or 2.5% normal sheep serum in PBS was applied for 1 h at room temperature to block nonspecific background staining. The sections were then incubated with following primary antibodies: monoclonal mouse anti-GFAP antibody (Sigma, St. Louis, Mo., U.S.A. diluted 1:500); polyclonal rabbit anti-caspase-3 p17 antibody (Cleaved Caspase-3 Antibody, detects only the large 17-20 kDa fragment of activated caspase-3, Cell Signaling Technology, USA, diluted 1:1000); mouse anti-PCNA antibody (DAKA, A/S, Denmark, diluted 1:100). After incubation with primary antibodies at 4° C. for 2 d (except for PCNA staining which was incubated overnight) the sections were incubated with biotinylated horse anti-mouse or goat anti-rabbit secondary antibody (1:200, Sigma) at 4° C. overnight. The ExtrAvidin (Sigma, 1:200), which had been prepared 1 h before use, was applied for 3 h at room temperature, and then reacted in 0.05% 3,3-diaminobenzidine (DAB) and PBS to produce a brown reaction product. Sections were dehydrated in a series of alcohols to xylene and coverslipped with mounting medium. Control sections were processed in the same way except the primary antibody was omitted from the incubation solution.

For specific visualization of microglia, isolectin B4 from Griffonia simplicifolia seeds (Sigma, St. Louis, Mo., U.S.A.) was used as a marker. The sections were pretreated with 1% H₂O₂ in 50% methanol for 30 min to quench the endogenous peroxidase activity after being deparaffinized. The sections were then incubated overnight at 4° C. with the iso-lectin primary antibody, diluted (1:4) in Tris buffered saline before being developed in DAB.

For TdT-mediated dATP nick end labeling (TUNEL) staining, the sections were pretreated for 15 min with Proteinase K (40 μg/ml; Sigma Chemical, St. Louis, Mo.), washed in PBS, then kept for 10 min with methanol containing 1% H₂O₂ to block non-specific peroxidase activity. Sections were then washed again in PBS and incubated for 5 min with TdT buffer (GIBCO-BRL, Life Technologies, Gaithersburg, Md.). DNA fragments were labeled with TdT and biotin-14-dATP (Gibco-BRL) for 1 h at 37° C. Subsequently, sections were washed in SSC buffer and incubated for 2 h with ABC reagent (Vector Laboratories). After washing, the sections were developed with DAB substrate. Sections were dehydrated in graded alcohols and mounted using DPX. A section that was pretreated with DNase 1 (Sigma Biosciences) to nick all DNA served as a positive control. A negative control slide was obtained with the omission of TdT from the incubation solution.

The number of caspase-3, TUNEL, GFAP, PCNA and isolectin B-4 positive cells were counted within the pyramidal layer of the CA1-2, CA3 and CA4 sub-regions on both sides of the hippocampus.

### Example 4

### Radioimmunoassay

The concentration of GPE in plasma and CSF were measured by a novel and specific double antibody radioimmunoassay (Batchelor et al. 2003; U.S. patent application Ser. No. 10/100,515, filed Mar. 14, 2002; and United States Utility Patent Application titled Anti-GPE Antibodies, Their Uses and Assays for Weakly Immunogenic Molecules, Gregory Brian Thomas, Bernhard Hermann Heinrich Breier and David Charles Batchelor inventors, filed concurrently (Attorney Docket No: NRNZ 1016 US2 DBB), each incorporated herein fully by reference). Prior to assay, the GPE samples, standards and tracer were derivatized with Bolton and Hunter reagent (Sigma-Aldrich, Sydney, Australia) to standardize the antibody binding configuration and maximize antibody recognition. The derivatized GPE radioimmunoassay shows complete parallelism with rat plasma and a recovery of unlabelled GPE added before assay of 83% (n=6 experiments). The ED-50 was 195 pg/tube, and the limit of detection was 2 pg/ml. The intra-assay CV was <10% over the range 0.5 to 25 ng/ml. Any samples reading off the standard curve were further diluted before being re-assayed.

### Example 5

### Statistical Analysis

Histological and immunohistochemical data were analyzed using two-way ANOVA followed by Bonferroni post-hoc tests for multiple comparisons, with brain regions treated as dependent factors. The levels of GPE in the CSF and plasma were analyzed using a one-way ANOVA. Data are presented as mean±SEM.

### Example 6

### GPE Pharmacokinetics

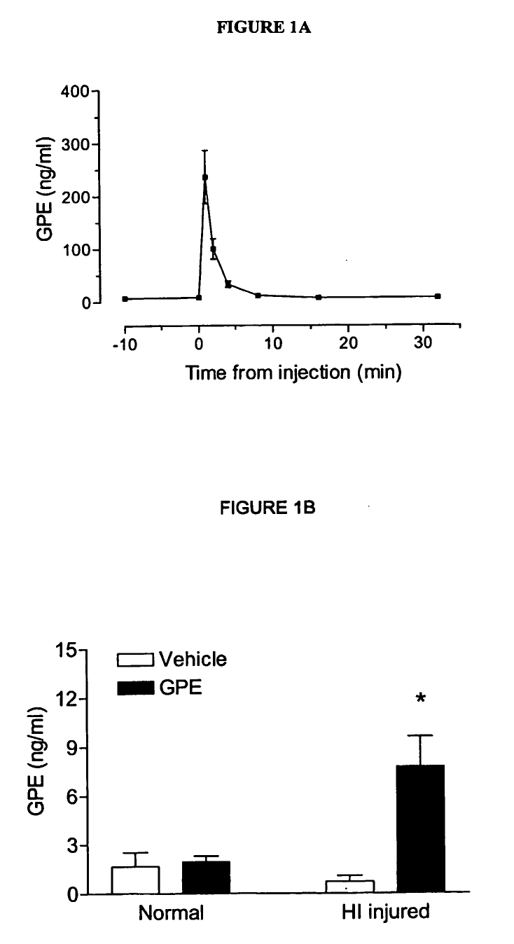

Following a single, 3 mg/kg i.v. injection of GPE in HI injured rats, plasma concentrations of GPE immediately increased from the baseline (8.1±4.1 ng/ml) to 236.6±50.0 ng/ml (FIG. 1A). The levels of GPE then rapidly declined back to baseline values (9.8±1.33 ng/ml) within the next 8 min. The half-life of GPE in plasma was estimated to be less than 2 min.

There was a significant increase (P<0.05) in GPE levels in the CSF following a continuous i.v. infusion of GPE (3 mg/kg/h for 4 h) in HI injured rats (7.75±1.87 ng/ml) compared with the vehicle treated HI control group (0.72±0.36 ng/ml, FIG. 1B). There was no difference in GPE levels in the CSF between GPE (1.94±0.36 ng/ml) and the vehicle (1.55±0.88 ng/ml) treated groups in non-HI injured rats.

### Example 7

### GPE Treatment Studies

HI brain injury resulted in severe neuronal injury in the ligated right hemisphere 4 days after HI injury (Table 2). Massive neuronal loss was seen in all sub-regions of the hippocampus. A mixture of selective neuronal loss, tissue pan-necrosis and cellular reaction were found in the cerebral cortex, all sub-regions of the hippocampus, the dentate gyrus and the striatum. There was no neuronal loss in the left hemisphere.

TABLE 2
GPE Bolus Plus Infusion vs i.v. GPE Bolus Alone

| | iv bolus 2 h post HI | | iv infusion with initial bolus | |
|---|---|---|---|---|
| | (15 mg/kg) | | (3 mg/kg) | |
| | Vehicle | GPE# | Vehicle | GPE*** |
| Striat | 1.30 ± 0.34 | 0.50 ± 0.20 | 2.52 ± 0.30 | 0.59 ± 0.24** |
| CA1-2 | 2.07 ± 0.50 | 0.85 ± 0.38 | 2.74 ± 0.29 | 0.62 ± 0.24** |
| CA3 | 1.89 ± 0.47 | 0.93 ± 0.41 | 2.56 ± 0.30 | 0.42 ± 0.18** |
| CA4 | 2.18 ± 0.53 | 1.11 ± 0.49 | 2.61 ± 0.32 | 0.58 ± 0.24** |
| DG | 1.64 ± 0.47 | 0.86 ± 0.43 | 2.04 ± 0.33 | 0.18 ± 0.14** |
| LC | 1.49 ± 0.45 | 0.52 ± 0.31 | 1.92 ± 0.31 | 0.21 ± 0.13** |

DG = dentate gyrus; LC = Lateral cortex
#p = 0.047;
*p < 0.01;
**p < 0.001;
***p < 0.0001

In animals injected with single i.v. bolus dose of 15 mg/kg GPE 2 h after the HI injury there was a modest reduction (overall P=0.047) in tissue damage scores compared with the vehicle treated group, with no difference between the groups in the individual brain regions (Table 2). In contrast, in animals injected with 3 mg/kg bolus followed by a continuous 3 mg/kg/h i.v. infusion of GPE 1-5 hours after HI injury (total dose 15 mg/kg), there was a highly significant reduction (overall P<0.0001) in the tissue damage scores when compared with the vehicle treated group. Post-hoc analysis showed that GPE treatment significantly reduced (P<0.01) the tissue damage in all of the brain regions examined.

GPE exhibited a broad effective dose range between 0.3-30 mg/kg/h (Table 3 below) when the 4 h iv infusion initiated at 1 h post injury without initial bolus iv injection.

TABLE 3
Dose Response to GPE Following Infusion Without the Initial Bolus

| | iv infusion 1-5 h post HI | | | | | |
|---|---|---|---|---|---|---|
| | 0.3 mg/kg/h | | 3 mg/kg/h | | 30 mg/kg/h | |
| | Vehicle | GPE* | Vehicle | GPE*** | Vehicle | GPE* |
| Striat | 1.87 ± 0.53 | 0.33 ± 0.33 | 1.20 ± 0.35 | 0.05 ± 0.05** | 1.83 ± 0.32 | 0.83 ± 0.27 |
| CA1-2 | 2.50 ± 0.49 | 0.78 ± 0.43 | 1.15 ± 0.40 | 0.16 ± 0.13 | 2.14 ± 0.50 | 1.68 ± 0.56 |
| CA3 | 2.36 ± 0.50 | 0.94 ± 0.54 | 1.19 ± 0.43 | 0.06 ± 0.06** | 2.04 ± 0.47 | 1.04 ± 0.41 |
| CA4 | 2.45 ± 0.57 | 0.94 ± 0.58 | 1.28 ± 0.46 | 0.09 ± 0.07* | 2.00 ± 0.53 | 1.08 ± 0.51 |
| DG | 1.86 ± 0.54 | 0.89 ± 0.59 | 0.97 ± 0.38 | 0.00 ± 0.00** | 1.29 ± 0.49 | 0.63 ± 0.36 |
| LC | 2.23 ± 0.51 | 0.93 ± 0.57 | 0.66 ± 0.34 | 0.00 ± 0.00** | 0.89 ± 0.46 | 0.13 ± 0.13 |

DG = dentate gyrus; LC = Lateral cortex
*p < 0.01;
**p < 0.001;
***p < 0.0001

TABLE 4
Efficacy of GPE Following Delayed Treatments

| | iv infusion 3-7 h post HI | | iv infusion 7-11 h post HI | |
|---|---|---|---|---|
| | (3 mg/kg/h | | (3 mg/kg/h) | |
| | Vehicle | GPE*** | Vehicle | GPE*** |
| Striatum | 1.62 ± 0.37 | 0.37 ± 0.25 | 2.60 ± 0.33 | 0.57 ± 0.25* |
| CA1-2 | 2.73 ± 0.41 | 0.54 ± 0.34** | 2.75 ± 0.51 | 0.65 ± 0.32** |
| CA3 | 2.77 ± 0.36 | 0.67 ± 0.39** | 2.71 ± 0.52 | 0.62 ± 0.29** |
| CA4 | 2.77 ± 0.45 | 0.58 ± 0.40** | 2.92 ± 0.47 | 0.38 ± 0.25** |
| DG | 1.69 ± 0.40 | 0.33 ± 0.33 | 3.00 ± 0.65 | 0.00 ± 0.00* |
| LC | 1.43 ± 0.42 | 0.02 ± 0.02* | 2.46 ± 0.48 | 0.06 ± 0.05* |

DG = dentate gyrus; LC = Lateral cortex
*p < 0.01;
**p < 0.001;
***p < 0.0001

No neuroprotective effects of GPE were observed with the 0.03 mg/kg/h dose (Table 3 above). GPE also reduced the injury when the treatment window was delayed either 3-7 h or 7-11 h after the injury (Table 4 above).

In the vehicle treated group, the tissue cavitations and atrophy were found within the ipsilateral hemisphere in the rats with severe brain damage 21 days after HI injury. The average tissue damage scores was 1.28±0.11 (n=6). Treatment with GPE (3 mg/kg/h 1-5 h post HI, without bolus) significantly reduced the tissue damage scores, with mild striatal damage in one of six rats treated with GPE (0.01±0.01, n=6, P<0.0001, FIG. 2A).

HI injury significantly increased the L/R ratio of the time taken to contact the patch (overall 2.45±0.51, P<0.0001, FIG. 2B) when compared to the normal control groups (1.05±0.06). Similar to our previous report (Guan et al. 2001a), the behavioral deficit was developed and maximized at day 3 followed by a spontaneous recovery at day 10 in the vehicle treated group. Treatment with GPE, 3 mg/kg/h 1-5 h post HI significantly reduced the L/R ratio of the time contact to the patch (1.08±0.07) compared to the vehicle treated group (2.45±0.51, P<0.01, FIG. 2B).

### Example 8

### Immunohistochemical Analysis

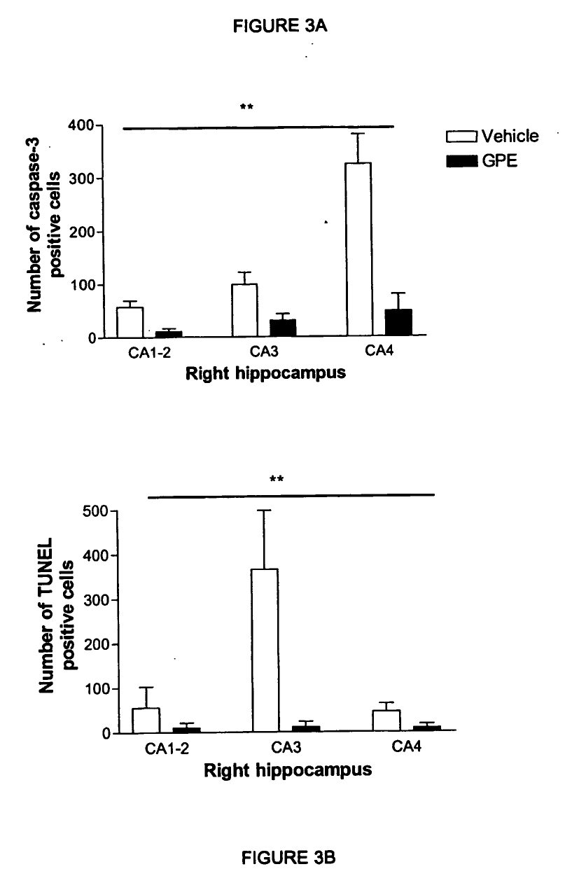

There were few caspase-3 positive cells observed in the control side of the hippocampus (average 18.9±3.9 cells, data did not show). HI brain injury resulted in an increase in caspase-3 positive cells in all sub-regions of ipsilateral (right) hippocampus (160.5±83.4 cells, FIG. 3A) compared to the control side of the hippocampus (18.9±3.9 cells). This increase in caspase-3 positive cells was more pronounced in the CA4 sub-region (325.5±55.2 cells). Treatment with GPE significantly reduced the number of caspase-3 positive cells in the hippocampus (29.9±10.6 cells, overall P<0.01), particularly in the CA4 sub-region of the hippocampus (47.7±31.8 cells, P<0.01) compared with the vehicle treated group (FIG. 3A). There were no TUNEL positive cells observed in the control side of the hippocampus. Similarly, HI injury resulted in an increase in the number of TUNEL positive cells detected in ipsilateral hippocampus (155.5±105.0 cells), particularly in the CA3 sub-region (365.5±132.6 cells, FIG. 3B) compared to the control side of the hippocampus. GPE treatment significantly reduced (P<0.01) the number of TUNEL positive cells in the hippocampus (10.6±0.7 cells), particularly in the CA3 sub-region of the hippocampus (11.7±11.7 cells) when compared with the vehicle treated group (FIG. 3B).

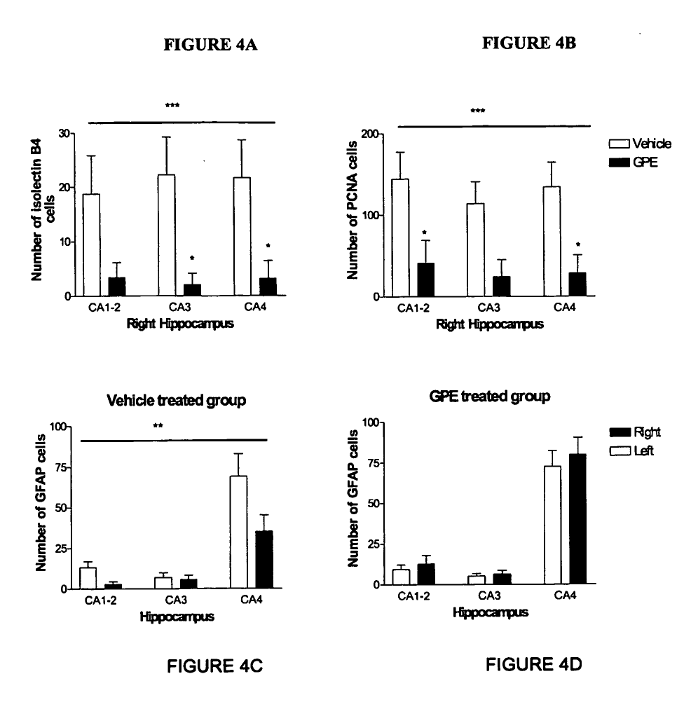

In response to HI injury there was an increase in the number of isolectin B-4 positive microglia in all the sub-regions of injured hippocampus (20.9±1.18 cells) compared to the control side, where isolectin B-4 positive cells were absent. Treatment with GPE significantly reduced (2.9±0.4 cells, overall P<0.0001) the number of isolectin B-4 positive cells, particularly in the CA3 and CA4 sub-regions (P<0.05) when compared with the vehicle treated group (FIG. 4A). The number of PCNA positive cells was increased in the ipsilateral hippocampus (130.9±8.9 cells) compared to the control side (1.6±0.7 cells, data did not show). Treatment with GPE significantly reduced (P<0.05) the number of PCNA positive cells in the ipsilateral hippocampus (30.9±5.1 cells, P<0.0001), particularly in the CA1-2 and CA4 sub-regions (P<0.05) when compared to the vehicle treated group (FIG. 4B). In vehicle treated animals, HI injury significantly reduced (P<0.05) the number of GFAP positive astrocytes in the ipsilateral hippocampus, particularly in the CA4 sub-region, when compared with the uninjured control hippocampus (FIGS. 4C and 4D). In contrast, there was no difference in the number of GFAP positive cells between the ipsilateral and contralateral hippocampus in the GPE treated group (FIGS. 4C and 4D).

This present study demonstrated that GPE produced robust and potent neuroprotective effects following continuous 4 h i.v. infusion in adult rats after HI brain injury. In contrast, following a single i.v. bolus administration of GPE showed only modest and sometimes variable effects. Following i.v. infusion the neuroprotective effects of GPE were global with a broad effective dose range from 0.3-30 mg/kg/h and extended treatment window of 7-11 h after HI injury. GPE infusion also achieved long-term neuroprotection, with improved somatosensory-motor function 20 days after injury. The neuroprotective effects of GPE in the hippocampus were associated with the inhibition of both caspase-3-dependent and -independent neuronal apoptosis. There was also evidence that GPE promoted the survival of astrocytes and suppressed the proliferation of microglial following ischemic injury.

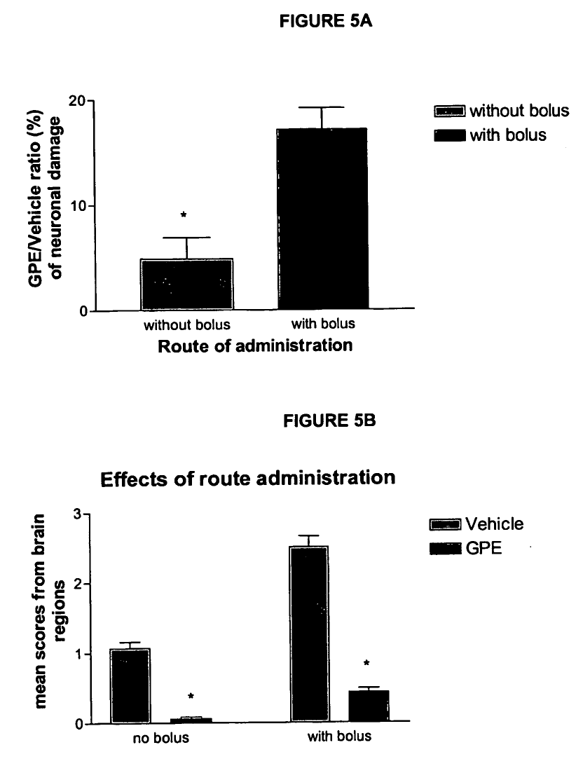

FIG. 5A depicts a graph comparing two GPE administration protocols; one involving GPE infusion (i.v.) after a prior bolus injection, and one involving GPE infusion without a prior bolus injection. In FIG. 5A, administration of GPE after a bolus (right column) resulted in a damage score (GPE/vehicle ratio) of nearly 20, whereas GPE administered as an infusion without prior bolus injection (left column) resulted in a lower neuronal damage score (GPE/vehicle ratio). Thus, we unexpectedly found that the typical “loading dose plus infusion” protocol was less effective in producing neuroprotection than GPE infusion alone, even though the total dose of GPE in the infusion alone protocol was less than that delivered using the bolus plus infusion protocol. This finding is even more surprising given the dose-related effects of GPE, which show an increase in effect of GPE with increasing dose over the range of from 0.3 mg/kg/h to 3 mg/kg/h. (Table 3).

FIG. 5B depicts a graph of brain damage scores for animals infused with vehicle or GPE without a bolus (left column of each pair) or after a bolus (right column of each pair).

The pharmaceutical industry generally has not yet identified neuroprotective compounds for treating ischemic brain injury (Fisher and Schaebitz, 2000) (Gladsrone et al. 2002). Although several forms of growth factors have been reported to be neuroprotective after various forms of ischemic brain injuries, their potential mitogenic effects and the difficulties in crossing the BBB have been well recognized limitations for the clinical development of growth factors, including IGF-1. Given that a small peptide will be more accessible to the CNS (Pardridge et al. 2002), drug development has now focused more on small molecules.

### Example 9

### Pharmacokinetic Study Design

Adult male Wistar rats weighing between 170 and 240 g were used. Animals were assigned to one of three treatment groups, each consisting of 6 animals per group. To facilitate intravenous bolus injections and blood sampling, all rats were surgically implanted with an indwelling jugular venous cannula under halothane anesthesia three days before the experiment.

Animals were given a single intravenous bolus injection of either 30 and 100 mg/kg GPE in 0.1 M succinate buffer (pH 6.5). Blood samples (≈220 ul) were collected into heparinized tubes containing 20 μl Sigma protease inhibitor cocktail for mammalian tissues diluted at a 1:9 ratio in 0.1 M PBS (pH=7). The samples were collected at 20, 10 and 0 min before and 1, 2, 4, 8, 16, 32 and 64 min after injection. The protease inhibitor cocktail used contains: 104 mM of AEBSF, 80 μM of Aprotinin, 2.1 mM of Leupeptin, 3.6 mM of Bestatin, 1.5 mM of Pepstatin A and 1.4 mM of E-64. The samples were centrifuged at 3000× g for 15 min at 4° C. and the plasma removed and stored at −80° C. until extraction and assay.

For a significant time after an intravenous bolus injection, GPE elimination followed a first-order reaction according to the equation: C=C₀e^{-kt}, where C represents GPE concentration in a particular time point, C₀ is the concentration when time (t) equals zero and k is the first-order rate constant expressed in units of concentration per hour. The k and half-life (t_{1/2}) were calculated from the slope of the linear regression line in the elimination phase of the semi-logarithmic plot of plasma concentration versus time as: Log C=−kt/2.3+log C₀. Results were expressed as mean±standard error.

Results

Radioimmunoassay Analysis

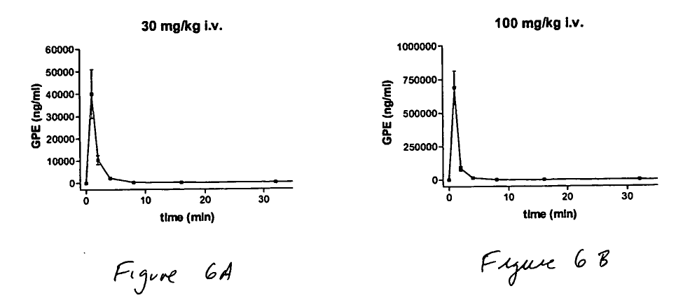

Following the development and validation of a specific and sensitive RIA for GPE, the pharmacokinetic properties of GPE were determined in vivo after i.v. administration. Plasma concentrations of GPE were markedly increased within 1 min after i.v. bolus injection (FIG. 6). After injection of 30 mg/kg (FIG. 6A) or 100 mg/kg (FIG. 6B) of GPE, peak concentrations of 40±10.8 and 689±125 μg/ml were observed. Plasma concentrations then rapidly declined in first-order reaction. The first order rate constant was calculated to be 0.15±0.014 (ng/ml/min, n=12), the half-life was calculated to be 4.95±0.43 (min, n=12) and the estimate clearance is 137.5±12.3 (ml/hr, n=12). We conclude that the clearance of GPE from the systemic circulation is extremely rapid with a very short half-life, and that stable concentrations of GPE in the circulation could be obtained by either repeated injection or by continuous infusion.

HPLC Analysis

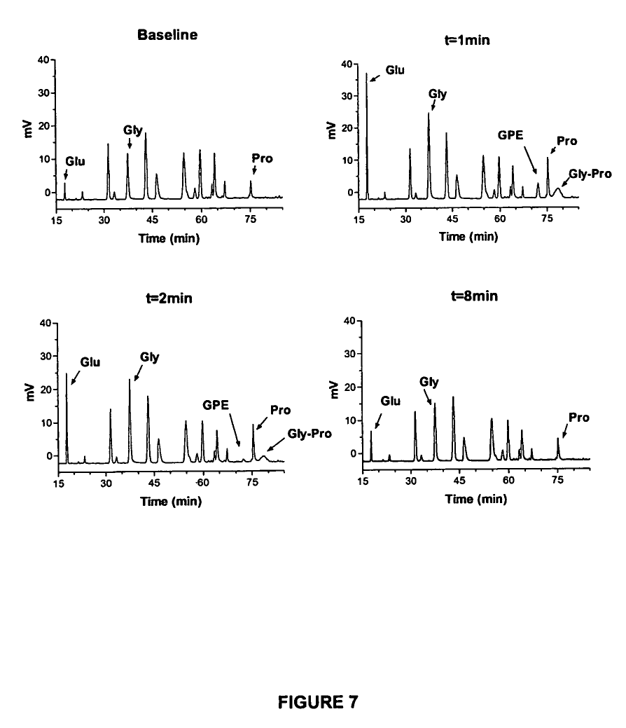

The pharmacokinetic data indicated a very rapid clearance of GPE from the rat circulation. We therefore investigated if the rapid clearance was from first pass clearance or was via a metabolic pathway. The HPLC elution profile shows that GPE elutes with a retention time of approximately 72 min (FIG. 7). The peak was sharp and clearly detectable above control plasma. No GPE was detected by HPLC in ‘unspiked’ native plasma.

The HPLC method employed was able to detect all potential metabolic products of GPE metabolism, Gly-Pro, Pro-Glu and the individual amino acids Glycine, Proline and Glutamate. At baseline the levels of Glu, Gly and Pro were 11, 31 and 31 μg/ml respectively. The GPE and Gly-Pro were below the limit of sensitivity for this HPLC method (300 ng/ml). At 1 min after an i.v. bolus injection of 30 mg/kg GPE, the levels of Glu, Gly and Pro had increased to 86, 62 and 80 μg/ml respectively (FIG. 7). GPE levels were 33 μg/ml and the dipeptide Gly-Pro could be identified as a broad peak with a level of 44 μg/ml. After 8 min the levels of all the metabolites and GPE had returned to baseline levels. Pro-Glu could not be identified in any sample.

Thus, we found that metabolic degradation of GPE occurs within the circulation, and that stable concentrations of GPE could be achieved by either repeated injection, continuous infusion, a combination of bolus injection and infusion, or via time-release methods.

### CONCLUSIONS

Certain embodiments of the present invention are directed to the use of a GPE assay to determine the pharmacokinetics of GPE in rats. In experiments in which we measured plasma GPE concentration following a single i.v. bolus of 30 and 100 mg/kg GPE, administration of GPE resulted in a significant but transient increase in plasma concentration of GPE, which during the decay period disappeared with a mean half-life of 4.95 min. The observed plasma half-life was totally unexpected based on prior studies demonstrating that GPE can be effective in decreasing cell degeneration or cell death in numerous conditions. We previously demonstrated that as little as a single injection of GPE can result in reduction in cell degeneration and/or death. Thus, the finding that the plasma half-life of GPE is about 2 to about 5 minutes under these conditions indicates that the therapeutic effects of GPE are potent. The half-life was not affected by the doses used but by individual variation from animals. The large variation in peak dose also reflects individual variation from animals and time necessary to obtain the sample.

The HPLC studies indicate that the proteolysis of GPE is via the formation of the dipeptide Gly-Pro, as Pro-Glu was not detected in any samples. This suggests that the metabolism of GPE initially involves the removal of the C-terminal glutamate followed by the rapid proteolysis of the remaining di-peptide, Gly-Pro, into its constituent amino acids. This finding also indicates that exopeptidases such as carboxypeptidases, peptidyldipeptidase, dipeptidases, metalloproteinases and aminopeptidases or other plasma enzymes can degrade the tripeptide in rats' plasma. This finding also indicates that co-administering GPE along with a peptidase or proteinase inhibitor can improve therapeutic effects of GPE, both by increasing the potency of the GPE and/or by prolonging the half-life of GPE in the animal.

GPE intravenous bolus injection followed first order kinetic and plasma samples collected from 12 rats. The elimination rate constant and half-life were determined from the slope of the linear regression line in the elimination phase of the semi-logarithmic plot of plasma concentration versus time. However, we couldn't obtain exactly Cmax at zero time due to rapid degradation of GPE by peptidases in plasma. Therefore the maximum concentration which we were able to get is at the 1 min time point which was lower than theoretical dose. As known, half-life is not influenced by dose used but by individual animals or other factors. The estimated clearances were calculated from rate constant multiplied by the estimated distribution volume. Another observation is that GPE is neuroprotective when it was given intraperitoneally which indicates GPE absorption could be influenced by other mechanism which have not yet been investigated (5).

Interestingly, the starved dwarf rats, (a control group outside of this study), had a basal level of GPE of approximately 2.5 ng/ml yet the fed Wistar rats had a basal level of approximately 10 ng/ml indicating that a low level of GPE is present in plasma. One potential source is the proteolytic cleavage of IGF-1 into des1-3 IGF-1 and the GPE by a protease in both brain and in serum.

Based on our current findings, continuous intravenous infusion is a useful mode of drug delivery for achieving and maintaining therapeutic blood levels of GPE. Repetitive intravenous bolus-dosing methods would require a possibly unrealistically short interval to achieve desired average plasma concentrations of GPE without producing large fluctuations in concentration. Similarly the level of proteolytic metabolites in plasma suggests that intraperitoneal or intramuscular injection would also result in significant loss of GPE before it can reach the site of action. A continuous intravenous infusion regimen can also allow for the treatment of both primary and secondary ischemic events that are encountered.

Other methods known in the art for producing stable concentrations of substances can also be used. For example, implantable “minipumps” (e.g., Alza Corporation) can be used to continuously infuse GPE at a desired location. In alternative embodiments, show-release compositions (e.g., carboxypolysaccharides/polyethylene oxide, polyethylene glycol, polylysine and the like, can be used to manufacture compositions for implantation and upon biodegradation of the matrix material, the GPE can be liberated and can have therapeutic effects. Such devices and compositions can be placed locally near the site to be treated (e.g., brain, spinal cord, peripheral nervous system) and can thereby produce sustained release of therapeutically active GPE.

We have developed new methods of in-tube sample preparation and analysis that takes advantage of the derivatization chemistry used in synthesizing hapten containing antigens to increase the sensitivity and specificity to the hapten to provide a useful, fast and novel approach for analysis for these small non antigenic peptides or molecules. In the past researchers have used Bolton and Hunter reagent to minimize the effects of iodination on the sensitivity of the assay. This, to our knowledge, is the first time that it has been used to increase the sensitivity of an assay for a target molecule. We have then used this method to identify for the first time the levels and clearance of GPE. Our data suggests that its probable route of administration for clinical trials is by intravenous infusion.

### Example 10

### Delayed Administration of GPE is Effective

Animal studies and surgery was carried out as described in Example 1.

Treatment Studies

Two separate studies of 14-16 HI injured rats and 14-16 control (vehicle treated) animals were carried out. In one study, HI injury was relatively mild, and in the other study, HI injury was severe. Each animal received a continuous 4 hour i.v. infusion of eithervehicle or 12 mg/kg GPE from 24-28 h after the HI injury. The rats were killed and the brains collected for histological analysis 4 days after the HI injury. Histological procedures were carried out as specified in Example 3. Statistical analysis was carried out as set out in Example 5.

Results

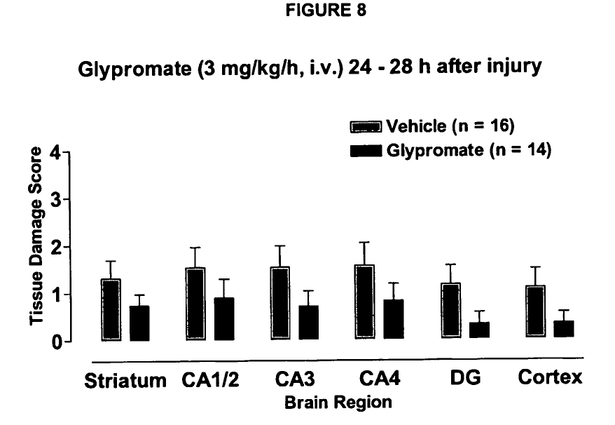

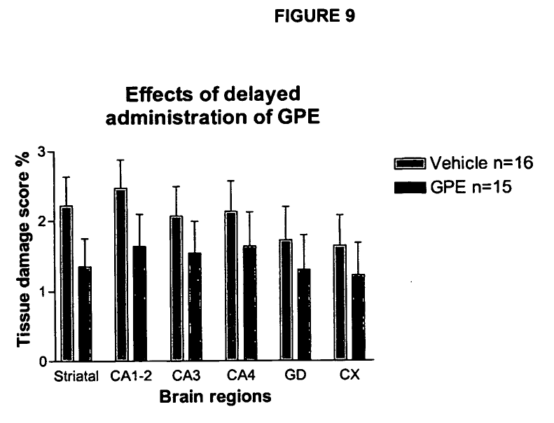

In each study, HI caused substantial neural damage, with the severely hypoxic animals (FIG. 9) having a damage score of between about 1.5 to about 2.5. In the other study (FIG. 8), the neural damage score was mild from about 1 to about 1.5.

GPE reduced the neuronal damage caused by HI in each group. In the mildly hypoxic animals, GPE caused a substantial and statistically significant reduction in damage score (FIG. 8). FIG. 8 depicts a graph of effects of GPE administered from 24 to 28 hours after HI injury. For each pair of columns, the left represents effects of vehicle and the right of each pair reflects effects of GPE. For each brain region studied, GPE infused during the time period resulted in significant neuroprotection.

In the severely hypoxic animals, GPE caused a reproducible decrease in damage score in each area of the brain studied (FIG. 9). However, in this study, the magnitude of HI injury was greater than that of the animals in FIG. 8. As with FIG. 8, in each brain region studied, GPE caused in increase in neuroprotection compared to vehicle controls. Thus, although the magnitude of the neuroprotection in FIG. 9 is somewhat less than that shown in FIG. 8, both of these studies indicated that GPE can have neuroprotective effects even if administered at 24 hours after injury.

So far most neuroprotective agents tested in animal models have less than a 3 h window of opportunity for treatment, and very few of them can be still effective when given within 6 h post initial injury. In contrast to GPE, other groups of agents with neuroprotective effects have a restricted delayed window of opportunity as early administration can worsen the outcome, e.g. vascular endothelial growth factor (VEGF). We have demonstrated that the effective window of opportunity for GPE is at least from 1-24 hrs post injury and pre-injury treatment has been also found effective in reducing brain injury. Based on these finding, the window of GPE is now the widest window known for treatment of neural injury.

On other hand, the degree of brain injury appeared to determine the window of opportunity for the treatment. It is known that neurons die more progressively when the injury is more mild, which provide a more extended window of opportunity for the treatment, whereas the window opportunity can be narrowed down when the neuronal injury is more severe. In general the animal models used for pre-clinical development are much more severe than human patients, therefore the mild brain injury may more represent human patients.

The examples and descriptions provided are intended to illustrate aspects of this invention and are not intended to be limiting. Other specific embodiments can be created by those of ordinary skill in the art and are all considered to be part of this invention. All publications cited herein are included in their entirety by reference.

### REFERENCES

Aberg, M. A., Aberg, N. D., Hedbacker, H., Oscarsson, J., and Eriksson, P. S. 2000. Peripheral infusion of IGF-I selectively induces neurogenesis in the adult rat hippocampus. Journal of Neuroscience 20, 2896-2903.

Auer, R. N., Kalimo, H., Olsson, Y., and Siesjö, B. K. 1985. The temporal evolution of hypoglycemic brain damage. I. Light- and electron-microscopic findings in the rat cerebral cortex. Acta Neuropathol. Berl. 67, 13-24.

Baskin, D. G., Wilcox, B. J., Figlewicz, D. P., and Dorsa, D. M. 1988. Insulin and insulin-like growth factors in the CNS. Trends in Neurosciences 11, 107-111.

Batchelor, D. C., Lin, H., Wen, J.-Y., Keven, C., van Zijl, P. L., Breier, B. H., Gluckman, P. D., and Thomas, G. B. 2003. Pharmacokinetics of glycine-proline-glutamate, the N-terminal tripeptide of insulin-like growth factor-1, in rats. Analytical Biochemistry 323, 156-163.

Beilharz, E. J., Williams, C. E., Dragunow, M., Sirimanne, E. S., and Gluckman, P. D. 1995. Mechanisms of delayed cell death following hypoxic-ischemic injury in the immature rat: evidence for apoptosis during selective neuronal loss. Brain Research 29, 1-14.

Brown, A. W. and Brierley, J. B. 1972. Anoxic-ischemic cell change in rat brain: light microscopic and fine structural observations. Journal of the Neurological Sciences 16, 59-84.

Cao, Y., Gunn A. J., Bennet, L., Wu, D., George, S., Gluckman P. D., Shao, X., and Guan, J. 2003. Insulin-like Growth Factor (IGF)-1 suppresses oligodendrocyte caspase-3 activation and increases glial proliferation after ischemia in near-term fetal sheep. J. Cereb Blood Flow Metab 23,739-747.

Coimbra, C., Drake, M., Boris-Moller, F., and Wieloch, T. 1996. Long-lasting neuroprotective effect of postischemic hypothermia and treatment with an anti-inflammatory/antipyretic drug. Evidence for chronic encephalopathic processes following ischemia. Stroke 27, 1578-1585.

Colbourne, F., Li, H., Buchan, A. M., and Clemens, J. A. 1999. Continuing postischemic neuronal death in CA1: influence of ischemia duration and cytoprotective doses of NBQX and SNX-111 in rats. Stroke 30, 662-668.

Donoghue, S., Baden, H. S., Lauder, I., Sobolewski, S., and Pringle, J. H. 1999. immunohistochemical localization of caspase-3 correlates with clinical outcome in B-cell diffuse large-cell lymphoma. Cancer Res. 59, 5386-5391.

Famularo, G., Polchi, S., and Panegrossi, A. 1998. Thrombolysis enters the race: a new era for acute ischemic stroke? Eur J Emerg. Med 5, 249-252.

Fisher, M. and Schaebitz, W. 2000. An overview of acute strike therapy. Arch Intern Med 160, 3196-3206.

Fujimura, M., Gasche, Y., Morita-Fujimura, Y., Massengale, J., and Kawase, M. 1999. Early appearance of activated matrix metalloproteinase-9 and blood-brain barrier disruption in mice after focal cerebral ischemia and reperfusion. Brain Research 842,92-100.

Galli, C., Meucci, O., Scorziello, A., Werge, T. M., Calissano, P., and Schettini, G. 1995. Apoptosis in cerebellar granule cells is blocked by high KCl, forskolin, and IGF-1 through distinct mechanisms of action: The involvement of intracellular calcium and RNA synthesis. Journal of Neuroscience 15, 1172-1179.

Gallyas, F., Hsu, M., and Buzsaki, G. 1992. Delayed degeneration of the optic tract and neurons in the superior colliculus after forebrain ischemia. Neuroscience Letters 144, 177-179.

Gasser, U. E., Weskamp, G., Otten, U., and Dravid, A. R. 1986. Time course of the elevation of nerve growth factor (NGF) content in the hippocampus and septum following lesions of the septohippocampal pathway in rats. Brain Research 376, 351-356.

Ginsberg, M. D. and Busto, R. 1989. Rodent models of cerebral ischemia. Stroke 20, 1627-1642.

Gladsrone, D. J., Black, S. E., and Hakim, A. M. 2002. Toward wisdom from failure—Lessons from neuroprotective stroke trials and new therapeutic directions. Stroke 33, 2123-2136.

Gluckman, P., Klempt, N., Guan, J., Mallard, C., Sirimanne, E., Dragunow, M., Klempt, M., Singh, K., Williams, C., and Nikolics, K. 1992. A role for IGF-1 in the rescue of CNS neurons following hypoxic-ischemic injury. Biochemical & Biophysical Research Communications 182, 593-599.

Gomez Pinilla, F., Lee, J. W., and Cotman, C. W. 1992. Basic FGF in adult rat brain: cellular distribution and response to entorhinal lesion and fimbria-fornix transection. Journal of Neuroscience 12, 345-355.

Guan, J., Gunn, A. J., Sirimanne, E. S., Tuffin, J., Gunning, M., Clark, R. G., and Gluckman, P. D. 2000. The window of opportunity for neuronal rescue with insulin-like growth factor-1 after hypoxia-ischemia in rats is critically modulated by cerebral temperature during recovery. Journal of Cerebral Blood Flow and Metabolism 20, 513-519.

Guan, J., Miller, O. T., Waugh, K. M., McCarthy, D., and Gluckman, P. D. 2001. Insulin-like growth factor-1 improves somatosensory function and reduces the extent of cortical infarction and ongoing neuronal loss after hypoxia-ischemia in rats. Neuroscience \tilde{ι}□, 299-306.

Guan, J., Waldvogel, H. J., Faull, R. L. M., Gluckman, P. D., and Williams, C. E. 1999. The effects of the N-terminal tripeptide of insulin-like growth factor-1, glycine-proline-glutamate in different regions following hypoxic-ischemic brain injury in adult rats. Neuroscience 89, 649-659.

Guan, J., Williams, C. E., Gunning, M., Mallard, E. C., and Gluckman, P. D. 1993. The effects of IGF-1 treatment after hypoxic-ischemic brain injury in adult rats. Journal of Cerebral Blood Flow and Metabolism 13, 609-616.

Guan, J., Williams, C. E., Skinner, S. J. M., Mallard, E. C., and Gluckman, P. D. 1996. The effects of insulin-like growth factor (IGF)-1, IGF-2, and Des-IGF-1 on neuronal loss after hypoxic-ischemic brain injury in adult rats—evidence for a role for IGF binding proteins. Endocrinology 137, 893-898.

Hsu, M., Sik, A., Gallyas, F., Horvath, Z., and Buzsaki, G. 1994. Short-term and long-term changes in the postischemic hippocampus. Annals of the New York Academy of Sciences 743, 121-139.

Jeon, B S., Jackson-lewis, V., and Burke, R E. 1995. 6-Hydroxydopamine lesion of the rat substantia nigra: time course and morphology of cell death. Neurodegeneration 4(2), 131-137.

Johnston, B. M., Mallard, E. C., Williams, C. E., and Gluckman, P. D. 1996. Insulin-like growth factor-1 is a potent neuronal rescue agent after hypoxic-ischemic injury in fetal lambs. Journal of Clinical Investigation 97, 300-308.

Klempt, N. D., Sirimanne, E., Gunn, A. J., Klempt, M., Singh, K., Williams, C. E., and Gluckman, P. D. 1992. Hypoxia-ischemia induces transforming growth factor beta 1 mRNA in the infant rat brain. Molecular Brain Research 13,93-101.

Kraig, R. P., Lascola, C. D., and Cagiano, A. 1995, Glial response to brain ischemia. In: Kettenmann, H. and Ransom, B. R. (Eds), Neuroglia New York. Oxford university press, pp. 964-976.

Lundgren, J., Smith, M. L., and Siesjö, B. K. 1992. Effects of dimethylthiourea on ischemic brain damage in hyperglycemic rats. Journal of the Neurological Sciences 113, 187-197.

Lutsep, H. L. and Clark, W. M. 1999. Neuroprotection in acute ischemic stroke. Current status and future potential. Drug R D 1, 3-8.

Markgraf, C. G., Kraydieh, S., Prado, R., Watson, B. D., Dietrich, W. D., and Ginsberg, M. D. 1993. Comparative histopathologic consequences of photothrombotic occlusion of the distal middle cerebral artery in Sprague-Dawley and Wistar rats. Stroke 24, 286-292.

Mary, V., Wahl, F., Uzan, A., and Stutzmann, J. M. 2001. Enoxaparin in experimental stroke: neuroprotection and therapeutic window of opportunity. Stroke 32, 993-999.

Matsui, T., Mori, T., Tateishi, N., Kagamiishi, Y., Satoh, S., Katsube, N., Morikawa, E., Morimoto, T., Ikuta, F., and Asano, T. 2002. Astrocytic activation and delayed infarct expansion after permanent focal ischemia in rats. Part I: enhanced astrocytic synthesis of s-100 beta in the periinfarct area precedes delayed infarct expansion. Journal of Cerebral Blood Flow and Metabolism 22, 711-722.

McNeill, H., Williams, C. E., Guan, J., Dragunow, M., Lawlor, P., Sirimanne, E., Nikolics, K., and Gluckman, P. D. 1994. Neuronal rescue with transforming growth factor-beta(1) after hypoxic-ischemic brain injury. NeuroReport 5, 901-904.

Nilsson-Håkansson, L., Civalero, I., Zhang, X., Carlsson-Skwirut, C., Sara, V. R., and Nordberg, A. 1993. Effects of IGF-1, truncated IGF-1 and the tripeptide Gly-Pro-Glu on acetylcholine release from parietal cortex of rat brain. NeuroReport 4, 1111-1114.

O'Donnell, S. L., Frederick, T. J., Krady, J. K., Vannucci, S. J., and Wood, T. L. 2002. IGF-I and microglia/macrophage proliferation in the ischemic mouse brain. GLIA 39, 85-97.

Pardridge, W. M. 2002. Drug and gene delivery to the brain: The vascular route. Neuron 36, 555-558.

Planas, A. M., Sole, S., and Justicia, C. 2001. Expression and activation of matrix metalloproteinase-2 and -9 in rat brain after transient focal cerebral ischemia. Neurobiol Dis 8, 834-846.

Sara, V. R., Carlsson-Sdwirut, C., Bergman, T., Jornvall, H., Roberts, P. J., Crawford, M., Hakansson, L. N., Civalero, I., and Nordberg, A. 1989. Identification of Gly-Pre-Glu (GPE), the aminoterminal tripeptide of insulin-like growth factor 1 which is truncted in brain, as a novel neuroaction peptide. Biochemical & Biophysical Research Communications 165, 766-771.

Sharp, F. R., Lu, A., Tang, Y., and Millhorn, D. E. 2000. Multiple molecular penumbras after focal cerebral ischemia. Journal of Cerebral Blood Flow and Metabolism 20, 1011-1032.

Snider, B. J., Gottron, F. J., and Choi, D. W. 1999. Apoptosis and necrosis in cerebrovascular disease. Annals of the New York Academy of Sciences 893, 243-253.

Springer, J. E., Nottingham, S. A., McEwen, M. L., Azbill, R. D., and Jin, Y. 2001. caspase-3 apoptotic signaling following injury to the central nervous system. Clin Chem Lab □ □, 299-307.

Tateishi, N., Mori, T., Kagamiishi, Y., Satoh, S., Katsube, N., Morikawa, E., Morimoto, T., Matsui, T., and Asano, T. 2002. Astrocytic activation and delayed infarct expansion after permanent focal ischemia in rats. Part II: suppression of astrocytic activation by a novel agent (R)-(−)-2-propyloctanoic acid (ONO-2506) leads to mitigation of delayed infarct expansion and early improvement of neurologic deficits. Journal of Cerebral Blood Flow and Metabolism 22, 723-734.

Thomas, G. B., Fairhall, K. M., and Robinson, I. C. A. F. 1997. Activation of the hypothalamo-pituitary-adrenal axis by the growth hormone(GH) secretagogue, GH-releasing peptide-6, in rats. Endocrine ι□ □, 1585-1591.

Velier, J. J., Ellison, J. A., Kikly, K. K., Spera, P. A., Barone, F. C., and Feuerstein, G. Z. 1999. Caspase-8 and caspase-3 are expressed by different populations of cortical neurons undergoing delayed cell death after focal stroke in the rat. Journal of Neuroscience 19, 5932-5941.

Wallace, M. C., Teasdale, G. M., and McCulloch, J. 1992. Autoradiographic analysis of 3H-MK-801 (dizocilpine) in vivo uptake and in vitro binding after focal cerebral ischemia in the rat. Journal of Neurosurgery 76, 127-133.

Williams, A. J., Hale, S. L., Moffett, J. R., Dave, J. R., Elliott, P. J., Adams, J., and Tortella, F. C. 2003. Delayed treatment with MLN519 reduces infarction and associated neurologic deficit caused by focal ischemic brain injury in rats via antiinflammatory mechanisms involving nuclear factor-kappaB activation, gliosis, and leukocyte infiltration. J Cereb Blood Flow Metab. 23, 75-87.

Yakovlev, A. G. and Faden, A. I. 2001. Caspase-dependent apoptotic pathways in CNS injury. Molecular Neurobiology □ □, 131-144.

Yamaguchi, F., Itano, T., Miyamoto, O., Janjua, N. A., Ohmoto, T., Hosokawa, K., and Hatase, O. 1991. Increase of extracellular insulin-like growth factor I (IGF-I) concentration following electrolytical lesion in rat hippocampus. Neuroscience Letters 128, 273-276.

Yamamoto, H. and Murphy, L. J. 1994. Generation of des-(1-3) insulin-like growth factor-I in serum by an acid protease. Endocrinology 135, 2432-2439.

Yamamoto, H. and Murphy, L. J. 1995. Enzymatic conversion of IGF-I to des(1-3)IGF-I in rat serum and tissues: A further potential site of growth hormone regulation of IGF-I action. Journal of Endocrinology 146, 141-148.

Yin, Q. W., Johnson, J., Prevette, D., and Oppenheim, R. W. 1994. Cell death of spinal motoneurons in the chick embryo following deafferentation: rescue effects of tissue extracts, soluble proteins, and neurotrophic agents. Journal of Neuroscience 14, 7629-7640.

We claim:

1. A method for protecting neuronal cells from degeneration in response to a neuronal insult, comprising intravenous administration to an animal in need thereof, a pharmaceutically effective amount of the tripeptide Gly-Pro-Glu (GPE).

2. The method of claim 1, wherein said GPE is administered as an infusion without a prior bolus injection.

3. The method of claim 1, wherein said GPE is administered as a bolus followed by an infusion.

4. The method of claim 1, wherein said GPE is administered from about 1 hours to about 24 hours after said neuronal insult.

5. A method for providing prolonged protection of neural cells from degeneration in an animal following a neuronal insult, comprising administering GPE to said animal via intravenous administration, said administration beginning from about 1 hour after the insult to about 24 hours after the insult.

6. A method of decreasing neural cell death or degeneration following a neuronal insult resulting from hypoxia/ischemia caused elective surgery, comprising administering GPE as an intravenous infusion over a duration of from about 1 to about 4 hours, said infusion beginning at a time from about 1 hour before to about 24 hours after said elective surgery.

7. A method of decreasing neural cell death or degeneration following a neuronal insult resulting from hypoxia/ischemia caused by stroke, comprising administering GPE as an intravenous infusion beginning at a time from about the onset of said stroke to about 24 hours after said stroke.

8. The method of claim 6, wherein said surgery is coronary artery bypass surgery.

9. The method of any of claims 1-8, wherein said GPE is administered in a pharmaceutically acceptable formulation.

10. The method of claim 3, wherein said GPE is administered as a bolus at a dose of about 0.03 mg/kg to about 30 mg/kg.

11. The method of any of claims 1-3, wherein said GPE is infused at a rate of about 0.03 mg/kg/h to about 30 mg/kg/h.

12. The method of any of claims 1-11, wherein said GPE is administered between about 1 to 7 hours after an insult that would otherwise result in neural degeneration or neuronal cell death.

13. The method of any of claims 1-11, wherein said GPE is administered between about 1 to about 11 h after an insult that would otherwise result in neural degeneration or neuronal cell death.

14. The method of claim 3, wherein the dose of GPE administered in said bolus is in the range of about 0.03 mg/kg to about 30 mg/kg, and wherein said GPE in said infusion is administered at a rate of about 0.03 mg/kg/h to about 30 mg/kg/h.

15. The method of claim 3, wherein the dose of GPE administered in said bolus is about 3 mg/kg and wherein said GPE in said infusion is administered at a rate of about 3 mg/kg/hr.

16. The method of claim 2, wherein said GPE is infused at a rate of about 0.3 mg/kg to about 3 mg/kg.

17. The method of any of claims 1-16, wherein said insult is a condition selected from the group consisting of Huntington's disease, Alzheimer's disease, Parkinson's disease, multiple sclerosis, amyotrophic lateral sclerosis, peripheral neuropathy, spinal muscular atrophy, Creutzfeldt-Jakob disease, AIDS dementia, progressive supranuclear palsy, myelinopathia centralis diffusa (vanishing white matter disease), chronic neurodegenerative disease, Down's syndrome, leukoencephalopathy, hypoxia, ischemia, coronary artery bypass graft (CABG) surgery and Schilder's disease, neuroblastoma, head injury, traumatic brain injury, stroke, reperfusion injury, epilepsy, toxin damage, radiation damage, asphyxia, an inflammatory condition, chronic or acute encephalomyelitis, encephalitis, optic neuritis, transverse myelitis, meningitis, panencephalitis, Devic's disease, progressive multifocal leukoencephalopathy, central pontine myelinolysis, depression, schizophrenia and neuromyelitis optica.

18. The method of any of claims 1-17, further comprising administering an additional neuroprotective agent.

19. The method of any of claims 1-18, wherein said additional neuroprotective agent is an anti-apoptotic agent or an anti-necrotic agent.

20. The method of claim 18, wherein said additional neuroprotective agent is selected from the group consisting of growth factors and associated derivatives (insulin-like growth factor-I [IGF-I], insulin-like growth factor-II [IGF-II], transforming growth factor-β1, activin, growth hormone, nerve growth factor, growth hormone binding protein, IGF-binding proteins [especially IGFBP-3], basic fibroblast growth factor, acidic fibroblast growth factor, the hst/Kfgk gene product, FGF-3, FGF-4, FGF-6, keratinocyte growth factor, androgen-induced growth factor, int-2, fibroblast growth factor homologous factor-1 (FHF-1), FHF-2, FHF-3 and FHF-4, keratinocyte growth factor 2, glial-activating factor, FGF-10 and FGF-16, ciliary neurotrophic factor, brain derived growth factor, neurotrophin 3, neurotrophin 4, bone morphogenetic protein 2 [BMP-2], glial-cell line derived neurotrophic factor, activity-dependant neurotrophic factor, cytokine leukaemia inhibiting factor, oncostatin M, an interleukin, α-interferon, β-interferon, γ-interferon, consensus interferon, TNF-α, clomethiazole; kynurenic acid, Semax, tacrolimus, L-threo-1-phenyl-2-decanoylamino-3-morpholino-1-propanol, adrenocorticotropin-(4-9) analogue [ORG 2766], dizolcipine [MK-801], selegiline, a glutamate antagonist selected from the group consisting of NPS1506, GV1505260, MK-801 and GV150526, an AMPA antagonist selected from the group consisting of 2,3-dihydroxy-6-nitro-7-sulfamoylbenzo(f)quinoxaline (NBQX), LY303070 and LY300164 and anti-inflammatory agent selected from the group consisting of an anti-MAdCAM-1 antibody and an antibody against an integrin α4β1 receptor and an integrin α4β7 receptor.

21. The method of claim 5, wherein said administration is carried out for a period of from about 1 to about 4 hours duration.

22. The method of any of claims 1-21, further comprising administration of an enzyme inhibitor capable of inhibiting the degradation of GPE in plasma.

23. The method of any of claims 1021, further comprising administration of one or more inhibitors of carboxypeptidases, aminopeptidases, peptidyldipeptidases, metalloproteinases and dipeptidases.

24. The method of any of claims 1-21, further comprising administration of one or more enzyme inhibitors selected from the group consisting of aprotinin, pepstatin A, bestatin, leupeptin, AEBSF and E-64.

25. A composition comprising:
GPE; and
at least one protease or peptidase inhibitor.

26. The composition of claim 26, wherein said inhibitor is capable of inhibiting a serine protease, a carboxypeptidase, an aminopeptidase, a cysteine protease, a dipeptidyl peptidase, a metalloprotease and/or a peptidyldipeptidase.

27. The composition of claim 25, wherein said at least one protease or peptidase inhibitor is selected from the group consisting of aprotinin, a metalloproteinase inhibitor, pepstatin A, bestatin, leupeptin, AEBSF and E-64.

28. The composition of any of claims 25-27 further comprising a pharmaceutically acceptable excipient.
